(19) United States
(12) Patent Application Publication
(10) Pub. No.: US 2010/0278311 A1
(43) Pub. Date: Nov. 4, 2010

(54) DEVICE AND METHOD FOR SCALING MEDICAL IMAGES

(76) Inventors: Kevin Hammerstrom, Richmond (CA); John Edward Aldrich, West Vancouver (CA)

Correspondence Address: DAVIS WRIGHT TREMAINE, LLP/Seattle, 1201 Third Avenue, Suite 2200, SEATTLE, WA, 98101-3045, US

(21) Appl. No.: 12/607,692

(22) Filed: Oct. 28, 2009

Related U.S. Application Data
(60) Provisional application No. 61/193,102, filed on Oct. 28, 2008.

(51) Int. Cl. A61B 6/00

(52) U.S. Cl. 378/163

(57) ABSTRACT

A radiographic image scaling device comprises a mounting unit and a reference object attached to the mounting unit. The reference object has a known actual size and shape and a sufficient radio-opacity to be visibly measurable in a radiographic image. The mounting unit has a radio-opacity that is different than that of the reference object, and a rigid body with at least one curved portion engagable with a surface for receiving a subject. The reference object is attached to a location on the mounting unit body that incremental movement of the mounting unit relative to the surface along the curved portion causes the reference object to incrementally move relative to the surface. Particularly, the reference object can be radio-opaque and the mounting unit can be is radiolucent.

### FIELD OF INVENTION

The present invention relates to a device and a method for scaling medical images, such as radiographic images.

### BACKGROUND OF THE INVENTION

The process of producing a medical image often results in magnification in the final image of the object that is being imaged. This is particularly true with modern digital imaging, such as Picture Archiving and Communication Systems (PACS) in which medical images are digitized and viewed on a screen. There are numerous medical applications in which the exact size of the original object needs to be known. For instance, in orthopedic applications such as hip or knee replacement surgeries, it is important that the exact size of the underlying structures such as bone be known. It is therefore important that the magnification of the medical image be known in order to calculate the true object size from the image. Magnification can vary depending on a wide range of factors.

One type of medical imaging process is radiographic imaging. Scaling of radiographic images can be achieved using radio-opaque objects of known size. Comparison of the size of the radio-opaque object in the radiographic image with the actual size of the radio-opaque object will give the magnification, or scale, of the image. This can then be used to calculate the true size of the objects being imaged, for instance bone structures, etc. As such, there is a need for devices and methods which allow easy and accurate scaling of radiographic images.

### SUMMARY OF THE INVENTION

According to one aspect of the invention, there is provided a radiographic image scaling device comprising a mounting unit and a reference object attached to the mounting unit. The reference object has a known actual size and shape and a sufficient radio-opacity to be visibly measurable in a radiographic image. The mounting unit has a radio-opacity that is different than that of the reference object, and a rigid body with at least one curved portion engagable with a surface for receiving a subject. The reference object is attached to a location on the mounting unit body such that incremental movement of the mounting unit relative to the surface along the curved portion causes the reference object to incrementally move relative to the surface. Particularly, the reference object can be radio-opaque and the mounting unit can be is radiolucent.

The mounting unit body can be a circular disc in which case the curved portion is the edge of the disc; the reference object is attached to the mounting unit off the disc's rotational axis. The circular disc can have a diameter of about 270 mm. Alternatively, the mounting unit body can comprise an elongated gripping member that is sized and shaped to be suitable for gripping by a human hand; the curved portion in this case is a curved surface of the gripping member, and the reference object is attached to one end of the mounting unit. The elongated gripping member can be a cylinder, in which case the reference object is mounted to the end of the mounting unit off the cylinder's rotational axis. Alternatively, the mounting unit can be an elliptical disc and in which case the curved portion is the edge of the disc.

The reference object can be a circular cylinder or disc that is mounted to or embedded in a surface of the mounting unit and have a predetermined diameter of about 25 mm.

The mounting unit can comprise markings on one or both of a front and a back surface of the mounting unit body; the markings are positioned to indicate the height of the reference object from the surface for receiving the subject when the curved portion is resting on the surface for receiving the subject.

The reference object can be made from a radio-opaque material selected from the group consisting of copper, aluminum, gold, platinum, palladium, silver, tungsten, bismuth-containing materials, alloys, metal oxides, barium-containing materials and radio-opaque contrast agents. The mounting unit can be made from radiolucent material selected from the group consisting of glass, wood, plastic and polymeric material.

According to another aspect of the invention, there is provided a method of scaling a radiographic image using the above scaling device. The method comprises: positioning a subject on a surface for receiving the subject and in a field of view of an imager; positioning the scaling device in the field of view of the imager and the curved portion of the scaling device on the surface for receiving the subject; adjusting the position of the reference object by moving the curved portion relative to the surface for receiving the subject such that the reference object generally corresponds to a desired imaging plane; radiographically imaging the subject and reference object and producing a resultant radiographic image; and measuring the size of the reference object in the radiographic image and determining the magnification of the image by comparing the measured size to the actual size of the reference object.

According to yet another aspect of the invention, there is provided a radiographic image scaling device comprising: a reference object having a known actual size and shape and a sufficient radio-opacity to be visibly measurable in a radiographic image; and a rigid elongated gripping member having a radio-opacity that is different than that of the reference object, a size and shape suitable for gripping by a human hand, and wherein the reference object is attached to one end of the elongated gripping member. The gripping member can comprise a curved portion engagable with a surface for receiving a subject, and the reference object can be attached to a location on the one end of the gripping member such that incremental movement of the gripping member relative to the surface along the curved portion causes the reference object to incrementally move relative to the surface. In particular, the gripping member can be a circular cylinder and the reference object is mounted on the distal end of the gripping member off the cylinder's rotational axis.

According to yet another aspect of the invention there is provided a method of scaling a radiographic image using the above-referenced scaling device having a rigid elongated gripping member. The method comprises: positioning a subject part of a patient in a field of view of an imager; positioning the scaling device such that the reference object generally corresponds to a desired imaging plane and is in the field of view of the imager; gripping the gripping member and pressing the one end of the gripping member having the reference object against the patient in the vicinity of the subject part; radiographically imaging the subject part and reference object and producing a resultant radiographic image; and measuring the size of the reference object in the radiographic image and determining the magnification of the image by comparing the measured size to the actual size of the reference object.

### BRIEF DESCRIPTION OF THE DRAWINGS

In the accompanying drawings, which illustrate one or more exemplary embodiments:

FIG. 1 is a schematic front view of a radiographic image scaling device comprising a radiolucent mounting unit and a radio-opaque reference object attached to the mounting unit, according to one embodiment of the invention.

FIG. 2 is a schematic front view of the scaling device shown in FIG. 1, positioned on a flat surface such that a “15” indicator on the mounting unit is positioned in a “12 O'Clock” position on the mounting unit relative to the surface.

FIGS. 3(a) to (d) illustrate a method of using the scaling device when taking a radiographic image of a body part of a patient.

FIG. 4 is a schematic illustration of an alternative embodiment of the scaling device, wherein the mounting unit is a elongated cylindrical gripping member.

FIGS. 5(a) and (b) illustrate a method of using the scaling device according to the alternate embodiment shown in FIG. 4 when taking a radiographic image of a body part of a patient.

### DETAILED DESCRIPTION OF EMBODIMENTS OF THE INVENTION

Directional terms such as “top”, “bottom”, “left”, “right”, “front”, “rear”, “clockwise” and “counter clockwise” are used in this description merely to assist the reader to understand the described embodiments and are not to be construed to limit the orientation of any described method, product, apparatus or parts thereof, whether in operation or in connection to another object.

As used herein, the term ‘medical imaging’, also commonly known as ‘clinical imaging’ refers to techniques and processes used to create images of the human body (or parts thereof) for clinical purposes such as medical procedures seeking to reveal, diagnose or examine disease or medical science including the study of normal anatomy and physiology. Medical imaging includes radiographic imaging (i.e. radiography) to produce radiographic images. Radiographic imaging generally uses electromagnetic radiation, in particular X-rays or gamma rays to produce images of body structures. Radiography may include, for instance tomography or computed tomography. The technique and processes described can be used for any x-ray imaging technique which comprises projection imaging, such as radiography, fluoroscopy, and angiography. The terms ‘radiography’ or ‘radiographic’ refer to any of these techniques.

The term “radio-opaque” (or ‘radiopaque’) refers to materials that inhibit or prevent the passage of electromagnetic radiation such as X-rays. Items that are radio-opaque may appear white on a radiographic image. The term “radiolucent” refers to materials that allow or are substantially transparent to the passage of electromagnetic radiation.

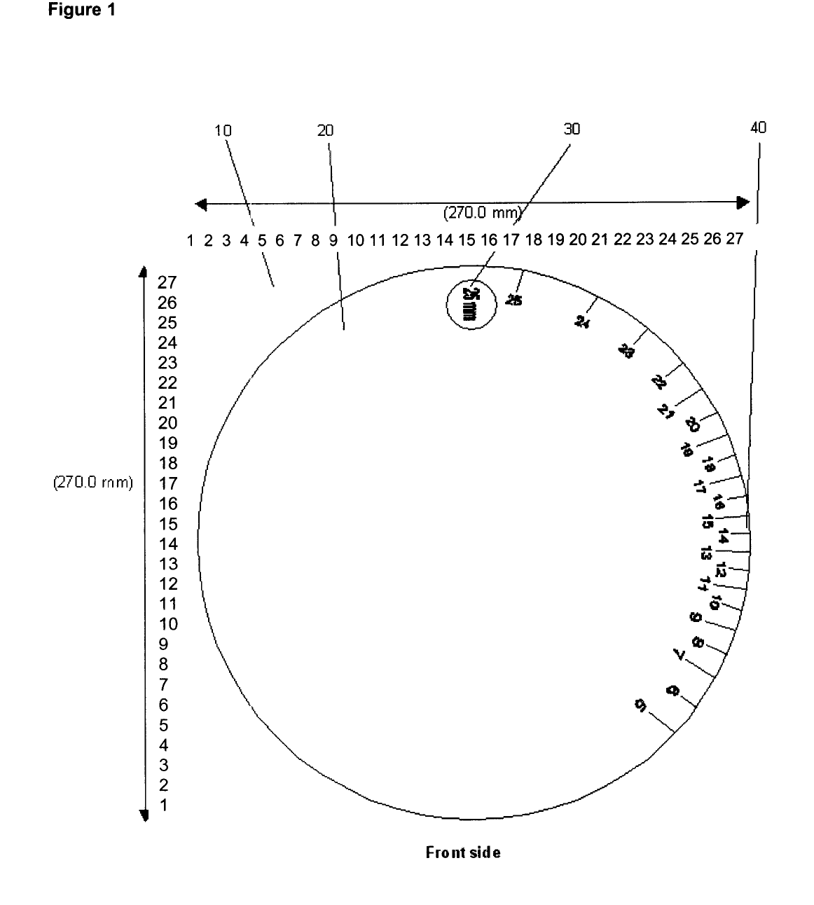

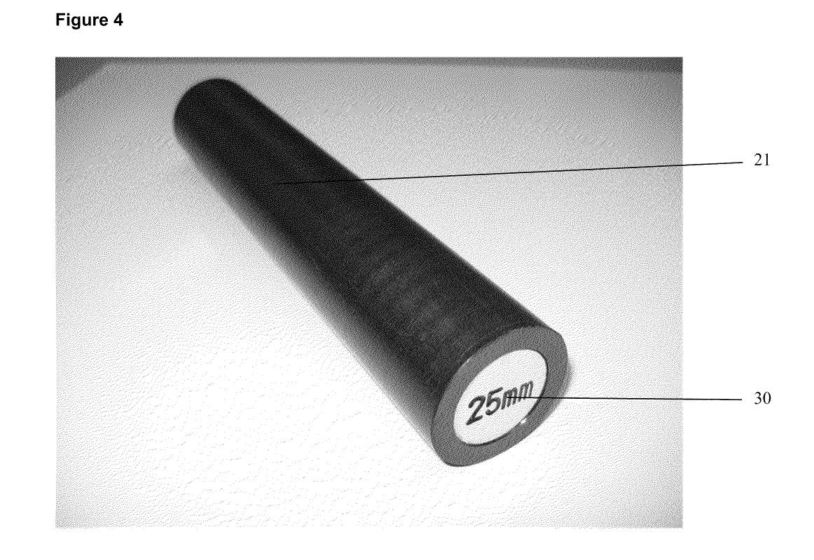

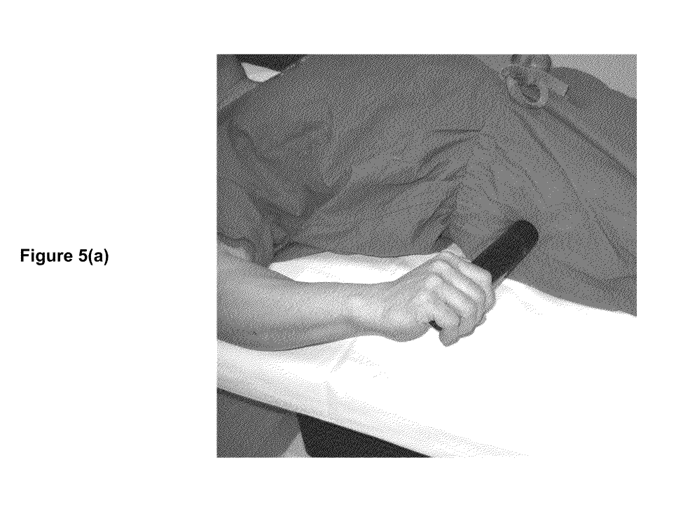

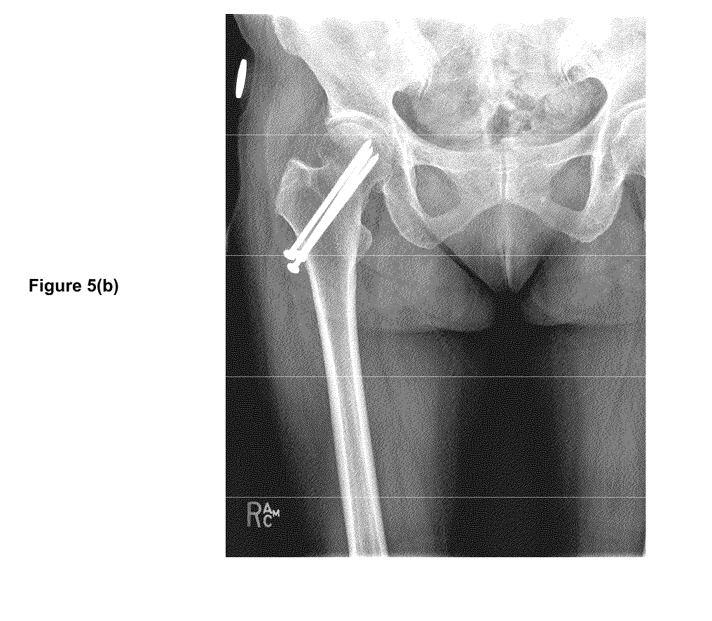

Medical images are often presented at a scale that is different from the original object being imaged. In many applications, for instance in orthopedic applications such as hip or knee replacement surgeries, it is important that the exact size of the objects such as bone and other underlying structures are known. Accurate scaling of medical images is thus important. The embodiments described herein relate to a scaling device for radiographic imaging and a method of using such a device to determine the size of an imaged subject. One embodiment of the radiographic imaging scaling device is shown in FIGS. 1 to 3, and another embodiment of the radiographic imaging scaling device is shown in FIGS. 4 and 5. In these described embodiments, the scaling device provides a method of accurately scaling subjects in a radiographic image. In these embodiments, the device comprises a reference object and a mounting unit on which the reference object is attached. The reference object has a known and defined size and shape and has a selected degree of radio-opacity and is preferably completely radio-opaque such that when the reference object is inserted into the field of view of a radiographic imager that is imaging a subject body part, that the resultant image of the body part will also show the reference object. The mounting unit has a body that is preferably substantially radiolucent, but can alternatively be partially radio-opaque so long as the mounting unit has a different degree of radio-opacity to that of the reference object. The mounting unit body is further preferably made from a single piece of material and is generally rigid and not flexible.

By positioning the reference object beside the subject and in the field of view of the imager, the size of the reference object can then be measured in the medical image and compared to the known actual size of the reference object in order to calculate the magnification of the image. The real size of the imaged subject body part can then easily be calculated. Thus, the reference object acts as a scaling device for the image subject.

One difficulty with image scaling using radio-opaque reference objects is that magnification of a reference object in an image can vary depending on the distance of the reference object from the detector plate of the imager; thus, it is important to place the reference object as close to the desired subject in order to maximize the accuracy of the scaling. To be more specific, it is important that the reference object be the same, or close to the same distance from a detector plate of the imager as the desired subject to be imaged. As will be described in more detail below, a portion of the mounting unit is curved to provide a user with a means of accurately and incrementally positioning the reference object sufficiently close to the subject body part that the reference object can provide a sufficiently accurate scale for measuring the size of the body part.

Referring now to FIGS. 1 to 3 and according to one embodiment of the invention, an adjustable radiographic image scaling device 10 comprises a generally radiolucent mounting unit 20 having a body in the shape of a thin circular disk and a generally radio-opaque reference object 30 in the shape of a circular cylindrical plug, and which is attached to the disk near the periphery thereof, and off-axis from the centre of the disk, i.e. off the rotational axis of the disc. The reference object 30 in this embodiment has a thickness less than or equal to that of the mounting unit 20 and is permanently embedded in a bore that extends through the mounting unit 20 such that the circular end surfaces of the reference object 30 are generally flush with the front and back surfaces of the mounting unit. However, the reference object 30 can alternatively be removably mounted to the mounting unit 20, and can alternatively be a thin disk that is permanently or removably mounted to the front or back surface of the mounting unit 20, or a thinner plug embedded in a cavity (not shown) in one of the surfaces of the mounting unit 30. A variety of methods may be used to secure the reference object 30 to the mounting unit 20 including, but not limited to: adhesives and other bonding agents, either permanent or temporary in duration, which can be applied to either the mounting unit 20 or the reference object 30 or both; hook and loop fasteners, and physical restraints integral to the curved mounting member such as: depressions in the surface. Also, more than one reference object (not shown) can be mounted to the mounting unit 20.

The reference object 30 is composed of a radio-opaque circular piece of metal selected from the group consisting of copper, aluminum, gold, platinum, palladium, silver, and tungsten. Alternatively, other non-limiting examples of suitable radio-opaque materials include bismuth-containing materials (e.g., bismuth trioxide, bismuth bicarbonate, bismuth oxychloride, and other bismuth-containing materials), metals (e.g., tungsten, tantalum, platinum, palladium, lead, gold, silver, titanium, and other metals), alloys (e.g., stainless steel, tungsten-containing alloys, tantalum-containing alloys, platinum-containing alloys, palladium-containing alloys, lead-containing alloys, gold-containing alloys, silver-containing alloys, titanium-containing alloys, and other alloys), metal oxides (e.g., titanium dioxide, zirconium dioxide, aluminum oxide, and other oxides), barium-containing materials (e.g., barium sulfate and other barium-containing materials), radio-opaque contrast agents (e.g., Omnipaque®, Renocal®, iodiamide meglumine, diatrizoate meglumine, ipodate calcium, ipodate sodium, iodamide sodium, iothalamate sodium, iopamidol, metrizamide, and other contrast agents). The reference object 30 can be made entirely out of a radio-opaque material, or be coated with such material.

The radio-opaque reference object 30 in this embodiment is a circular cylinder or disk having a diameter of 25 mm. The diameter and shape are deliberately selected and used in the method of determining the size of a body part in a radiographic image as will be discussed below. While other shapes and sizes can be selected for the reference object 30, a circular shape is particularly useful as the diameter dimension remains the same regardless of the orientation of the reference object to an imager's detector (not shown), and a 25 mm diameter is sufficiently large enough to be visible and accurately measurable in radiographic images of human body parts. The thickness of the reference object 30 is not essential to the method of determining the size of a body part in a radiographic image, but should be thick enough that the radiographic object 30 can be seen in the radiographic image when placed edge-wise relative to the imager.

The mounting unit 20 is made of a radiolucent plastic that is sufficiently strong and relatively inflexible to be handled by a person during radiographic imaging, and can be easily cleaned and sterilized with conventional liquid agents. Alternatively, other materials having these properties can be used for the mounting unit 20. In particular, the mounting unit 20 can be made from any material which is essentially transparent to x-ray radiation, such as glass, wood or, preferably, plastic or polymeric material. Although the entire mounting unit 20 need not be radiolucent, the area transparent to x-ray radiation must be large enough to not interfere with the full use of the device 10; that is, the mounting unit 20 should not have any radio-opaque material that would block or otherwise interfere with the imaging of the reference object 30 in the medical image.

The mounting unit 20 has a body which in this embodiment is a thin circular disk having a diameter of 270 mm and thickness of 6 mm. This shape and diameter is found to be particularly convenient for imaging human body parts, and in particular is large enough to be easily grasped and positioned by a patient or health care professional against the body part to be imaged, and yet thin enough to unobtrusively position against the body part to be imaged, e.g. between the legs for measuring the hip, pelvis or legs. Alternately, the device 10 could be easily placed to the side of the body for measuring the outer hip, shoulder, etc. The circular shape is particularly useful as this allows the patient or health care professional to accurately and incrementally adjust the reference object 30 relative to the body part to be imaged, and relative to the plane of the imager. Rotation of the circular disk causes a change in the position of the reference object 30 relative to a surface A on which the device 10 is placed (such surface also being the surface which the patient sits or lies). The curved shape of the mounting unit 20 provides the ability to incrementally adjust the position of the reference object 30. In order to facilitate this, the reference object 30 should be mounted away from the center of the disk and in this embodiment is mounted at the peripheral edge of the front surface of the mounting unit 20. Rotation of the scaling device 10 would thus move the reference object 30 around the center axis of the mounting unit 20 and would cause the reference object 30 to move up or down in the field of view of the imager.

While the mounting unit 20 body in this embodiment is a circular disk, the mounting unit 20 bodies can have other shapes, and preferably have a shape that has at least some curved portions that can be placed against the surface A to allow for accurate and incremental adjustment of the mounting unit 20 relative to a body part. One suitable alternate shape for the mounting unit 20 body is an ellipse, and in particular an elliptical disk or elliptical cylinder.

Dimensional markings 40 are provided along part of the edge of the front and back surfaces of the mounting unit 20 which indicate the height of the reference object 30 relative to the surface A on which the mounting unit 20 is placed. The markings 40 comprises lines and associated numbers, and are engraved or painted on the front and back surfaces of the mounting unit 20 in a different color than the mounting unit 20 for ease of reading. Alternatively, the markings 40 can be applied to only one of the front and back surfaces, and can be instead painted, inlayed, cut, broken, drilled, bored, punctured, machined, stained, etched, textured, carved or otherwise applied to the mounting unit 20. The font size of the numerical markings 40 is approximately 7-8 mm, and the numeric text “5” to “25” in single digit increments indicate the height in centimeters of the reference object 30 from the surface A and is placed on the mounting unit 20 so as to be easily distinguishable from adjacent text. An additional marking “25 mm” is provided on the reference object 30 and provides the user with the diameter of the reference object 30 which will be used in the method of determining the size of an imaged subject. These markings 40 may be situated at the edge of the circular mounting unit 20, allowing the patient or health care professional to easily view them and use them as a reference point when placing the reference object 30. For instance, markings 40 that are at the edge of the device 10 may be situated such that the marking 40 that is at the top of the mounting unit disk 20 in a particular orientation represents the distance from the center of the reference object 30 to the part of the mounting unit disk 20 contacting the surface A.

The angular distances between markings 40 provide a relationship between angular position of the mounting unit 20 and the linear distance (height) of the reference object 30 to the surface A when the mounting unit 20 is placed edgewise and perpendicular to the surface A. The following angular distances shown in Table 1 are determined from an imaginary line running through the centre of the mounting unit disk 20 and the center of the reference object 30 in a clockwise direction on the front side of the mounting unit disk 20 and in a counterclockwise direction on the back side of the mounting unit disk 20:

TABLE 1

| Marking (in cm) | Angle (degree) |
|---|---|
| 25 | 12.9 |
| 24 | 27.1 |
| 23 | 36.4 |
| 22 | 43.9 |
| 21 | 50.5 |
| 20 | 56.6 |
| 19 | 62.2 |
| 18 | 67.6 |
| 17 | 72.7 |
| 16 | 77.8 |
| 15 | 82.7 |
| 14 | 87.6 |
| 13 | 92.4 |
| 12 | 97.3 |
| 11 | 102.2 |
| 10 | 107.3 |
| 9 | 112.4 |
| 8 | 117.8 |
| 7 | 123.4 |
| 6 | 129.5 |
| 5 | 136.1 |

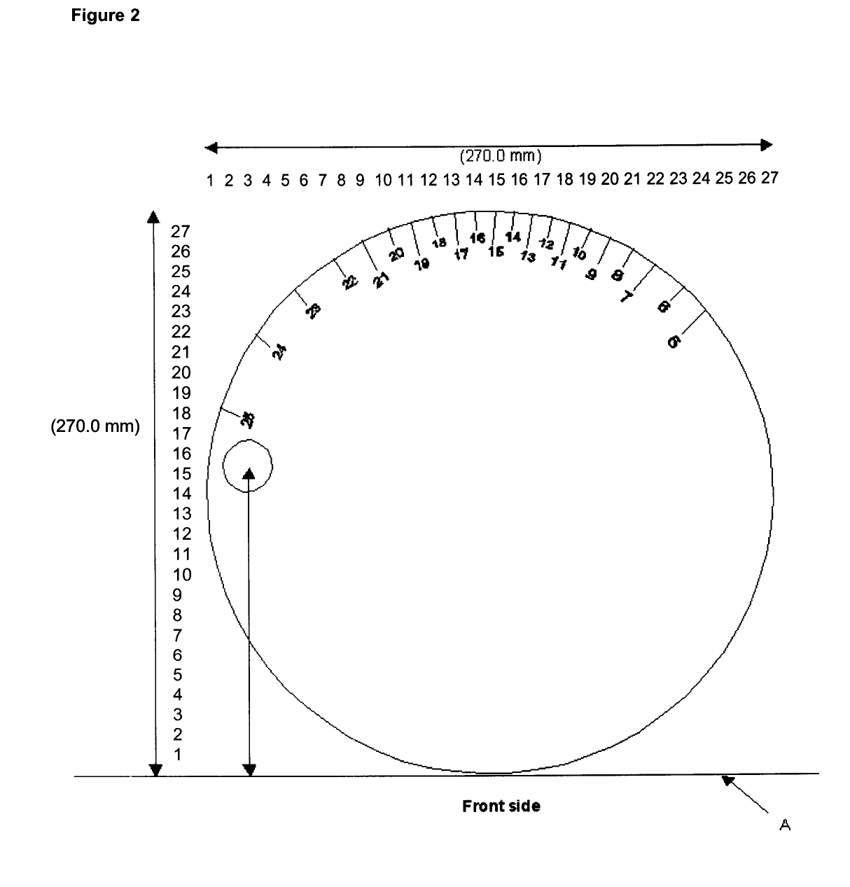

Referring to FIG. 2, these markings 40 can be used to determine the height of the reference object 30 to the surface A by determining which marking is at the “12 O'clock” position on the mounting unit 20 when the reference object 30 is in the desired position. In FIG. 2, it can be seen that the reference object 30 is about 15 cm above the surface A.

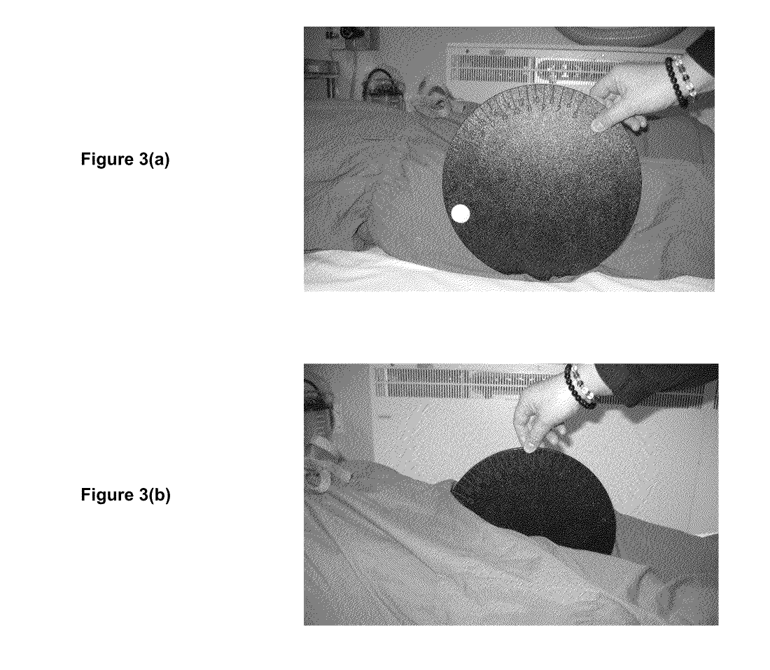

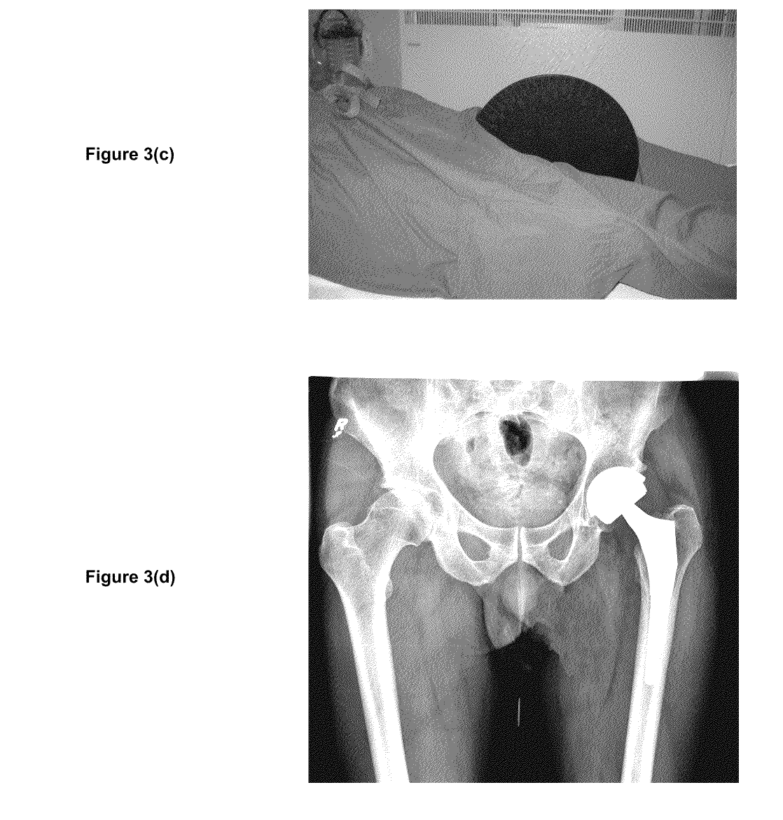

Referring now to FIGS. 3(a) to (d), a method of using the scaling device 10 in determining the size of a subject in a radiographic image is now described in respect to a patient who is having his pelvis and femur bones imaged. First, a patient sits or lies on a surface and a health care professional palpates for the bone plane of the bone(s) to be imaged. Then, a person holds the scaling device 10 such that it rests on the surface and the radiographic object 30 is at the height of the bone plane. Note is made of the marking 40 in the 12 o'clock position, which provides the height of the reference object 30 to the surface (see FIG. 3(a)). The patient sits or lies on the surface such that the body part to be imaged is in the field of view of the imager, and the scaling device 10 is also placed in the field of view of the imager with the noted marking in the same 12 o'clock position, thereby ensuring that the reference object 30 remains in the bone plane (see FIG. 3(b) wherein the scaling device is placed between the patient's legs). The patient then holds still for the radiographic exposure. The health care professional ensures that the scaling device 10 does not rotate after being positioned into place (see FIG. 3(c)) and verifies that after the exposure was taken and prior to removing the device from the field of view that the physical and angular position of the scaling device was maintained during exposure, to ensure reliability of the magnification factor. After the exposure has been taken, the resultant image can be reviewed to ensure that the reference object 30 is visible and completely within the field of view. Once this is confirmed, the diameter of the reference object 30 is measured in the image, and divided by the actual diameter of the reference object 30 to determine the scaling or magnification factor. This factor is then used to measure the size of the bones that were imaged.

In a preferred embodiment the scaling device 10 is held in place by the patient without any additional fastening means. However it is understood that the device can be held in place by a number of ways including by friction, VELCRO fasteners, adhesives, or any of a variety of fastening means such as clamps or clips.

Referring now to FIG. 4 and according to an alternate embodiment of the present invention, the mounting unit 20 has a body that is a single rigid elongated gripping member 21. The reference object 30 is attached to one end of the gripping member. The gripping member 21 is sized and shaped to be easily and comfortable gripped by a human hand. The gripping member 21 may have a textured surface that allows for a firm grip by a human hand. The gripping member 21 is further made from a material that is rigid to allow the user to exert enough pressure to place the end of the gripping member 21 with the reference object against patient's body and in close proximity to the subject body part to be imaged. In a preferred embodiment the gripping member 21 is made of Acrylonitrile butadiene styrene (ABS). The gripping member 21 may be held in the hand of the user to allow for close placement of the radio-opaque reference object 30 to the desired region to be imaged.

In one specific embodiment the gripping member 21 is an elongated circular cylinder having a circular end surface on which the reference object 30 is attached. In another embodiment the gripping member 21 is an elongated elliptical cylinder. In yet another alternate embodiment (not shown), the gripping member 21 is shaped to conform to the user's hand, and in particular has finger grip indentations molded or otherwise formed into the surface of the gripping member 21.

In one embodiment (not shown), the reference object 30 is embedded on peripheral edge of one end surface of the gripping member 21 (“reference object end”). Therefore, the gripping member can be placed on a surface A and rotated to incrementally adjust the height of the reference object 30 relative to the surface A in the same manner as the first embodiment described above. Markings can be provided along part of the edge of the distal end surface to aid the user in determining the suitable position of the reference object 30. Alternatively, and as shown in FIG. 4, the reference object is mounted in the center of the cylindrical gripping member; in this embodiment, rotation will not affect the height of the reference object 30 relative to the surface but the user can position and hold the reference object 30 in the desired bone plane by pressing the device 10 firmly against the patient body; in some cases, the device 10 may be lifted off the surface A on which the patient sits.

In a preferred embodiment the reference object 30 is permanently embedded in a bore in the center of the end surface of the cylindrical gripping member 21. The circular end surface of the reference object 30 is generally flush with the distal end surface of the cylindrical gripping member 21.

The gripping member 21 is preferably formed form a single rigid piece of material to provide sufficient structural strength for the device to be placed against a patient's body with sufficient pressure to hold the device in place while the radiographic image is being taken. Being able to firmly grip the rigid gripping member 21 and apply pressure against a patient's body allows for close placement of the reference object not only at the bone plane, but also as close as possible to the desired region to be imaged, thereby improving accuracy of the scaling. This is of particular importance in the obese population and may result in lower x-ray exposure to the individual by reducing the number of x-rays required to completely image the region of interest.

Referring to FIGS. 5(a) to (b), a method of using the alternate embodiment of the scaling device 10 shown in FIG. 4 is described. The patient is instructed to hold the gripping member 21. The health care professional palpates for the bone plane and places the gripping member 21 so that the reference object 30 is at the same height as the bone plane; this may result in the device 10 being placed on the surface on which the patient sits or be raised from the surface. The patient holds the gripping member 21 in the desired position, determined by the health care professional, so that the reference object is in the field of view of the imager; in FIG. 5(a), the body part to be imaged is the pelvis, and the patient presses the gripping member 21 tightly against the hip into the tissue overlying the bone of interest during the exposure. After the exposure is taken, the resultant image is reviewed to ensure that the reference object 30 is clearly visible and completely within the field of view.

While particular embodiments have been described in the foregoing, it is to be understood that other embodiments are possible within the scope of the invention and are intended to be included herein. It will be clear to any person skilled in the art those modifications of and adjustments to this invention, not shown, are possible without departing from the spirit of the invention as demonstrated through the exemplary embodiment.

What is claimed is:

1. A radiographic image scaling device comprising:
(a) A reference object having a known actual size and shape and a sufficient radio-opacity to be visibly measurable in a radiographic image; and
(b) a mounting unit having a radio-opacity that is different than that of the reference object, and a rigid body with at least one curved portion engagable with a surface for receiving a subject, wherein the reference object is attached to a location on the body such that incremental movement of the mounting unit relative to the surface along the curved portion causes the reference object to incrementally move relative to the surface.

2. The device of claim 1 wherein the reference object is radio-opaque and the mounting unit is radiolucent.

3. The device of claim 1, wherein the mounting unit body is a circular disc and the curved portion is the edge of the disc, and the reference object is attached to the mounting unit off the disc's rotational axis.

4. The device of claim 1 wherein the mounting unit body comprises an elongated gripping member sized and shaped to be suitable for gripping by a human hand, wherein the curved portion is a curved surface of the gripping member, and the reference object is attached to one end of the mounting unit.

5. The device of claim 4 wherein the elongated gripping member is a cylinder, and the reference object is mounted to the end of the gripping member off the cylinder's rotational axis.

6. The device of claim 1 wherein the mounting unit is an elliptical disc and the curved portion is the edge of the disc.

7. The device of claim 1, wherein the reference object is a circular cylinder that is embedded in a surface of the mounting unit.

8. The device of claim 7, wherein the reference object has a predetermined diameter of about 25 mm.

9. The device of claim 1, wherein the mounting unit comprises markings on one or both of a front and a back surface of the mounting unit, the markings positioned to indicate the height of the reference object from the surface for receiving the subject when the curved portion is resting on the surface for receiving the subject.

10. The device of claim 3, wherein the mounting unit has a diameter of about 270 mm.

11. The device of claim 1, wherein the reference object is made from a radio-opaque material selected from the group consisting of copper, aluminum, gold, platinum, palladium, silver, tungsten, bismuth-containing materials, alloys, metal oxides, barium-containing materials and radio-opaque contrast agents.

12. The device of claim 1, wherein the mounting unit is made from radiolucent material selected from the group consisting of glass, wood, plastic and polymeric material.

13. A method of scaling a radiographic image using a scaling device comprising a reference object having a known actual size and shape and a sufficient radio-opacity to be visibly measurable in a radiographic image; and a mounting unit having a radio-opacity that is different than that of the reference object and a rigid body with at least one curved portion engagable with a surface for receiving the subject, the reference object being attached to a location on the mounting unit body such that incremental movement of the mounting unit relative to the surface along the curved portion causes the reference object to incrementally move relative to the surface; the method comprising:
(a) positioning a subject on the surface for receiving the subject and in a field of view of an imager;
(b) positioning the scaling device in the field of view of the imager and the curved portion of the scaling device on the surface for receiving the subject;
(c) adjusting the position of the reference object by moving the curved portion relative to the surface for receiving the subject such that the reference object generally corresponds to a desired imaging plane;
(d) radiographically imaging the subject and reference object and producing a resultant radiographic image; and
(e) measuring the size of the reference object in the radiographic image and determining the magnification of the image by comparing the measured size to the actual size of the reference object.

14. A radiographic image scaling device comprising:
(a) a reference object having a known actual size and shape and a sufficient radio-opacity to be visibly measurable in a radiographic image; and
(b) a rigid elongated gripping member having a radio-opacity that is different than that of the reference object, a size and shape suitable for gripping by a human hand, and wherein the reference object is attached to one end of the elongated gripping member.

15. The device of claim 14, wherein the gripping member comprises a curved portion engagable with a surface for receiving a subject, and the reference object being attached to a location on one end of the gripping member such that incremental movement of the gripping member relative to the surface along the curved portion causes the reference object to incrementally move relative to the surface.

16. The device of claim 15, wherein the gripping member is a circular cylinder and the reference object is mounted on the end of the gripping member off the cylinder's rotational axis.

17. A method of scaling a radiographic image using a scaling device comprising a reference object having a known actual size and shape and a sufficient radio-opacity to be visibly measurable in a radiographic image and a rigid elongated gripping member having a radio-opacity that is different than that of the reference object, the gripping member having a size and shape suitable for gripping by a human hand, and wherein the reference object is attached to one end of the elongated gripping member, the method comprising:
(a) positioning a subject part of a patient in a field of view of an imager;
(b) positioning the scaling device such that the reference object generally corresponds to a desired imaging plane and is in the field of view of the imager;
(c) gripping the gripping member and pressing the one end of the gripping member having the reference object attached thereto against the patient in the vicinity of the subject part;
(d) radiographically imaging the subject part and reference object and producing a resultant radiographic image; and
(e) measuring the size of the reference object in the radiographic image and determining the magnification of the image by comparing the measured size to the actual size of the reference object.
